SEQUENCE LISTING (8 sequences)

SEQ ID NO: 1 - protein, 27 aa, Artificial Sequence
  Synthetic peptide based on p53 protein
GEYFTLQIRGRERFEMFRELNEALELK

SEQ ID NO: 2 - protein, 32 aa, Artificial Sequence
  Synthetic peptide based on p53 protein
GEYFTLQIRGRERFEMFRELNEALELKDAQAG

SEQ ID NO: 3 - protein, 35 aa, Artificial Sequence
  Synthetic peptide based on p53 protein
RKKRRQRRGEYFTLQIRGRERFEMFRELNEALELK

SEQ ID NO: 4 - protein, 40 aa, Artificial Sequence
  Synthetic peptide based on p53 protein
RKKRRQRRGEYFTLQIRGRERFEMFRELNEALELKDAQAG

SEQ ID NO: 5 - protein, 47 aa, Artificial Sequence
  Synthetic peptide based on p53 protein
KRALPNNTSSSPQPKKKPLDGEYFTLQIRGRERFEMFRELNEALELK

SEQ ID NO: 6 - protein, 52 aa, Artificial Sequence
  Synthetic peptide based on p53 protein
KRALPNNTSSSPQPKKKPLDGEYFTLQIRGRERFEMFRELNEALELKDAQAG

SEQ ID NO: 7 - protein, 27 aa, Artificial Sequence
  Synthetic peptide based on p53 protein
GRKKRRQRRGEYFTLQIRGRERFEMFR

SEQ ID NO: 8 - protein, 37 aa, Artificial Sequence
  Synthetic peptide based on p53 protein
GRKKRRQRRRGEYFTLQIRGRERFEMFRELNEALELK

(19) United States
(12) Patent Application Publication
(10) Pub. No.: US 2021/0353784 A1
(43) Pub. Date: Nov. 18, 2021

(54) DETECTING AND TREATING CANCERS USING CELL PENETRANT MTP53-OLIGOMERIZATION-DOMAIN PEPTIDE

(71) Applicant: Research Foundation of the City University of New York, New York, NY (US)

(72) Inventors: Jill Bargonetti-Chavarria, Bronx, NY (US); Gu Xiao, New York, NY (US); Brian Zeglis, New York, NY (US); George Annor, New York, NY (US); Kimberly Fung, New York, NY (US)

(21) Appl. No.: 17/318,706

(22) Filed: May 12, 2021

Related U.S. Application Data
(60) Provisional application No. 63/023,306, filed on May 12, 2020.
(60) Provisional application No. 63/186,409, filed on May 10, 2021.

(51) Int. Cl. A61K 51/08; A61K 47/64; A61K 49/00

(52) U.S. Cl. CPC A61K 51/088; A61K 49/0032; A61K 49/0056; A61K 47/64

(57) ABSTRACT

Peptide formulations of a general formula of X-mtp53ODP, where mtp53ODP is a peptide that binds to tetrameric mp53 tetramers in vivo. X is a detection agent, such a fluorophore or radioligand, and/or a DNA-damaging agent. The primary structures of suitable mtp53ODPs are given as SEQ ID NOS: 1-8.

### CROSS-REFERENCE TO RELATED APPLICATIONS

This application claims priority to, and is a non-provisional of, U.S. Patent Applications 63/023,306 (filed May 12, 2020) and 63/186,409 (filed May 10, 2021), the entirety of which are incorporated herein by reference.

### STATEMENT OF FEDERALLY SPONSORED RESEARCH OR DEVELOPMENT

This invention was made with Government support under grant number U54 CA221704(5) awarded by the National Cancer Institute and grant numbers R01 CA239603, R01 CA240963, U01CA221046, R01CA204167 and R01CA239603 from the National Institute of Health. The government has certain rights in the invention.

### REFERENCE TO A SEQUENCE LISTING

This application contains a Sequence Listing in computer readable form. The computer readable form is incorporated herein by reference.

### BACKGROUND OF THE INVENTION

The subject matter disclosed herein relates to compositions and methods for detecting and/or treating cancer and more particularly, to compositions and methods for detecting and/or treating cancer by targeting mutated p53 proteins (mtp53).

Breast cancer is the most common cancer among women and is the second leading cause of death from cancer among women. Approximately 30-35% of invasive primary breast cancers have mutated p53. Mutant p53 proteins (mtp53) are stabilized specifically in tumors, which is the key requisite for its gain of functions (GOF) activities such as proliferation, migration, invasion, survival, metabolism, chemoresistance, and tissue architecture that are associated with cancer development. The p53 is mutated in approximately 80′% of patients with the triple negative breast cancer (TNBC) (lack of detectable Estrogen Receptor (ER), Progesterone Receptor expression (PR) and HER2 gene amplification). Therefore, the high frequency of p53 mutations in TNBC suggests therapeutic strategies ought to be used for detecting mutant p53. To date, no single therapeutic or diagnostic strategy has been found to be entirely satisfactory. Accordingly, alternative strategies are desired.

The discussion above is merely provided for general background information and is not intended to be used as an aid in determining the scope of the claimed subject matter.

### SUMMARY

This disclosure provides peptide formulations of a general formula of X-mtp53ODP, where mtp53ODP is a peptide that binds to tetrameric mp53 tetramers in vivo. X is a detection agent, such a fluorophore or radioligand, and/or a DNA-damaging agent. The primary structures of suitable mtp53ODPs are given as SEQ ID NOS: 1-8.

In a first embodiment, a composition of matter is provided. The composition of matter comprising: a peptide with a formula of X-mtp53ODP wherein X is selected from a group consisting of (1) a detection agent, (2) a DNA-damaging agent and (3) combinations thereof, wherein X is covalently bonded to mtp53ODP which is a peptide selected from a group consisting of: GEYFTLQIRGRERFEMFRELNEALELK (SEQ ID NO: 1); GEYFTLQIRGRERFEMFRELNEALELKDAQAG (SEQ ID NO: 2); RKKRRQRRGEYFTLQIRGRERFEMFRELNEALELK (SEQ ID NO: 3); RKKRRQRRGEYFTLQIRGRERFEMFRELNEALELKDAQAG (SEQ ID NO: 4); KRALPNNTSSSPQPKKKPLDGEYFTLQIRGRERFEMFRELNEALELK (SEQ ID NO: 5), KRALPNNTSSSPQPKKKPLDGEYFTLQIRGRERFEMFRELNEALELKDAQAG (SEQ ID NO:6); GRKKRRQRRGEYFTLQIRGRERFEMFR (SEQ ID NO: 7); and GRKKRRQRRRGEYFTLQIRGRERFEMFRELNEALELK (SEQ ID NO: 8).

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1A is a schematic of the structure of Cy5p53Tet, a fluorophore Cy5 conjugated mtp53-oligomerization-domain peptide (mtp53ODP).

FIG. 1B shows live cell imaging staining of MCF7 and MDA-MB-468 cells after 2 h of incubation with 500 nM Cy5p53Tet (red). Hoechst staining (blue) was used to stain the nuclei. Two independent experiments with biological replicates were performed.

FIG. 1C depicts p53 protein levels in MCF7 and MDA-MB-468 cells determined by Western blot analysis before carrying out live cell imaging.

FIG. 1D is a graph showing quantification of Cy5p53Tet uptake in MCF7 and MDA-MB-468 cells via Nikon Element analysis. At least 200 cells per sample were measured by fluorescence microscopy.

FIG. 1E depicts the results of flow cytometry of MCF7 and MDAMB-468 cells after incubation with 100 or 500 nM Cy5p53Tet for 2 h at 37° C. FlowJo software was used to analyze the cytometric data.

FIG. 1F is a bar graph demonstrating geometric MFI from the FACS experiments in FIG. 1E.

FIG. 1G shows a MTT assay conducted in MCF7, MDA-MB-468, HCC70, SK-BR-3, and MCF10A cells to measure mitochondrial dehydrogenase activity in response to 500 nM Cy5p53Tet treatment for 24 h. Three independent experiments with biological replicates were performed for all ±SEM *p-value ≤0.05, **p-value ≤0.01, ***p-value ≤0.001.

FIG. 2A depicts results of a Western blot analysis showing ER and p53 protein levels in MCF7 and MDA-MB-468 cells performed before implantation.

FIG. 2B shows in vivo optical imaging of Cy5p53Tet uptake in mice bearing bilateral MCF7 and MDA-MB-468 xenograft models. A representative image acquired 30 min after injection is shown. The tumor is marked by a “T.”

FIG. 2C is an image showing epifluorescence imaging of MCF7 and MDA-MB-468 tumors excised 80 min after the administration of Cy5p53Tet.

FIG. 2D depicts epifluorescence intensity quantification of the tumors resected at 40, 80, and 180 min after injection.

FIG. 2E illustrates the results of a SDS-PAGE analysis of extracts collected from the tumors in the in vivo imaging experiment. Protein (25 μg) from each sample was run on a 12% polyacrylamide gel, and the fluorescence signal from Cy5p53Tet was interrogated and quantified. The expression levels of p53 and MDM2 in the same tumor samples were determined by Western blot analysis.

FIG. 3A shows (top) the results of live cell imaging of Cy5p53Tet (red) in MDA-MB-468 shp53 cells with or without shRNA induction. Cells were imaged by confocal microscopy after 30 min, 2 h, 4 h, and 24 h incubation of 500 nM Cy5p53Tet. Hoechst staining (blue) was used to stain the nucleus. Three independent experiments with biological replicate were performed. A representative picture acquired after 2 h of incubation is shown. (Bottom) Mtp53 protein levels in MDA-MB-468 shp53 cells with or without shRNA induction were determined by Western blot analysis before carrying out live cell imaging. Whole-cell extract (50 μg) was loaded on 10% SDS-PAGE gel.

FIG. 3B depicts quantification of Cy5p53Tet uptake in MDA-MB-468 shp53 cells with or without mtp53 depletion. *p-value ≤0.05, **p-value ≤0.01, ***p-value ≤0.001.

FIG. 3C shows the results of co-immunoprecipitation assay carried out with purified mtp53 R273H and Cy5p53Tet in a molar ratio of 1:1. Anti-p53 DO1 antibody-coupled magnetic beads were used to pull down Cy5p53Tet/mtp53 complex, and normal mouse IgG-coupled magnetic beads were used as a control.

FIG. 4A shows a Western blot carried out to detect mtp53 oligomer using anti-p53 DOJ antibody.

FIG. 4B shows Cy5p53Tet fluorescence signal detected using the Cy5 channel.

FIG. 4C shows Merged mtp53 (green) and Cy5p53Tet (red) image demonstrating a high-molecular-weight signal (yellow, indicate with arrow) that is likely an mtp53/Cy5p53Tet complex.

FIG. 4D illustrates Cy5p53Tet treated with glutaraldehyde without MDA-MB-468 cell lysate.

FIG. 5A shows Cy5p53Tet peptide perturbs gain of function of mtp53 in TNBC PDX models with mtp53. In vivo optical imaging of Cy5p53Tet peptide uptake in bilateral PDX WHIM6/WHIM25 xenograft models. 2 million WHIM6 cells and WHIM25 cells were injected subcutaneously on the left and right shoulders of each mouse. 10 nmol Cy5p53Tet peptide was intravenously injected to each mouse when tumor size reached to 100-200 mm³. At 1 hr, 2 hr and 3 hr post Cy5p53Tet peptide injection, the tumors were removed and the epifluorescence images were taken using an IVIS Spectrum. Representative epifluorescence imaging of excised WHIM6 and WHIM25 tumors 2 hr post Cy5p53Tet peptide injection was shown. Cy5p53Tet peptide epifluorescence intensity quantification in WHIM6 and WHIM25 tumors was analyzed.

FIG. 5B shows Cy5p53Tet peptide signal in tumor tissue generated by extracting proteins from the WHIM6 and WHIM25 tumors 1 hr, 2 hr and 3 hr post injection of Cy5p53Tet peptide. 25 μg of proteins were run on 12% polyacrylamide gel and the Cy5p53Tet peptide accumulated in tumors was scanned at Cy5 channel and quantified. The intensity of Cy5p53Tet peptide was analyzed by ImageJ. p53 and MDM2 protein levels from the WHIM6 and WHIM25 tumors post-injection with Cy5p53Tet peptide at 1 hr, 2 hr and 3 hr were determined by Western blot analysis. 25 ug of proteins were load on 12% SDS-PAGE gel.

FIG. 5C is a Western blot analysis showing p53, p63 and p73 protein levels from the WHIM6 and WHIM25 tumors 2 hr post injection with Cy5p53Tet peptide. 75 ug of proteins were load on 10% SDS-PAGE gel (left panel). p53, PARP1 and p73 protein levels from the WHIM6 and WHIM25 tumors 1 hr, 2 hr and 3 hr post injection with Cy5p53Tet peptide were determined by Western blot analysis (right panel). 50 ug of proteins were load on 10% SDS-PAGE gel.

FIG. 5D shows live cell imaging staining of MDA-MB-468, 184A1 and MCF10A cells with Cy5p53Tet peptide was shown in red. Cells were imaged by confocal microscopy after 2 hr incubation of 500 nM of Cy5p53Tet peptide. Hoechst staining was used to stain the nucleus and was shown in blue.

FIG. 6 is a sequence alignment of several of the disclosed mtp53ODP peptides showing alignment by the first residue in the p53TD Q strand.

FIG. 7 quantifies the results of live cell imaging staining of MDA-MB-468 and MCF7 cells with 4 h incubation of 500 nM Cy5 conjugated first (TAT-mtp53ODP-35mer, SEQ ID NO: 3) and second-generation (TAT-mtp53ODP-27mer (SEQ ID NO: 7) and TAT-mtp53ODP-37mer (SEQ ID NO: 8)) after 4 h incubation. Cells were imaged by confocal microscopy. Quantification of Cy5 conjugated TAT-mtp53ODP uptake in MDA-MB-468 cells and MCF7 cells was conducted by Nikon Element analysis. Relative region of interest (ROI) intensity values showed a statistically significant higher TAT-mtp53ODP uptake in MDA-MB-468 cells than MCF7 cells and significant higher TAT-mtp53ODP uptake than scramble peptide in both MDA-MB-468 and MCF7 cells. Three independent experiments with biological replicate were performed. Statistical analyses were conducted in Graphpad Prism 9. Results are expressed as mean+SEM. Statistical significance for hypothesis testing was performed by Student's t-test. The following format was used to assign significance based on P-value: *p-value ≤0.05, ** p-value ≤1 0.01, **** p-value ≤0.001.

### DETAILED DESCRIPTION OF THE INVENTION

This disclosure provides methods and compositions for detecting and killing cancer cells that express oncogenic mutant p53 (mtp53) proteins. Over 70% of all cancers over-express mutant p53 proteins. The p53 protein has five functional domains: a transactivation domain within the N-terminal region (TAD, residues 1-42), a proline-rich domain with a pro-apoptotic role (PRD, residues 63-97), a sequence-specific DNA binding domain (DBD, residues 98-292), an oligomerization domain which confers the tetrameric structure necessary for p53 function (TD, residues 325-355), and a highly basic C-terminal domain (CTD, residues 363-393) which interacts with DNA in a sequence non-specific manner. The p53TD has a β-strand (GIu326-Arg333), a tight turn (Gly334), and an α-helix (Arg335-Gly356). Two monomers form a dimer through their antiparallel β-sheets and α-helices and two dimers become a tetramer through the formation of a four-helix bundle. Wild type p53 binds to DNA as a homotetramer mediated by the p53 tetramerization domain (TD).

This disclosure provides mtp53-oligomerization-domain peptides (mtp53ODP), a family of peptides derived from p53 tetramerization domain. The disclosed methods utilize the biomarker mtp53 as a therapeutic and/or diagnostic vehicle (i.e. it is a mtp53 theranostic). The methods relate to using p53 oligomerization-domain peptides to detect cancers with stable mtp53 and also to directly target such cancers for DNA damage-mediated cell death. The disclosed methods also relate to noninvasive imaging and treating disorders associated with high levels of mtp53 protein expression.

The peptides described herein provide several advantages for imaging and targeting pan-cancers with over-expression of mtp53. The ability to non-invasively image cancers expressing the critical biomarker mtp53 at the whole-body level has never been achieved. With the present technology it is possible to separate subjects into appropriate target groups and determine the response of mtp53 expressing cancers to different treatment modalities. Moreover, the peptides can be linked to variable imaging, and cell killing with a connected radioligand, and moieties including those for positron emission tomography (PET) imaging for preclinical and clinical settings. In addition to the advantage for non-invasive imaging the peptides listed can all be used as agents in vivo for both imaging and targeted cell killing with different combinations of targeting moiety mtp53ODP.

These mtp53ODPs include peptide SEQ ID NOS: 1-8, plus a moiety X, which may be a detection agent (such as a radioligand or a fluorophore), a DNA-damaging agent radioligands or a moiety that functions as both a detection agent and a DNA-damaging agent. The formulation is any form of X-mtp53ODP.

In one embodiment, X is a detection agent such that the composition has a formula such as ⁸⁹Zr-mtp53ODP, ¹⁸F-mtp53ODP for PET/CT imaging, FL-mtp53ODP for fluorescent dye labeled non-radioactively labeled derivatives (like Cy5p53Tet) that can be used for fluorescent labeling prior to surgery to increase tumor visibility. Cyanine fluorophores other than Cy5 (e.g. Cy3, Cy7, etc.) may also be used.

In one embodiment, X is a tumor-targeting moiety for the delivery of radiation such as ¹³¹I-mtp53ODP for therapeutic radiation treatment and click chemistry based ¹⁷⁷Lu-labeled tetrazine (Tz) radioligands for pretargeted radiotherapy and SPECT imaging.

Methods for administering agents such as X-mtp32ODP patients are known. For example, a dilute solution may be administered as an intravenous drip.

Herein is a description of variants of the mtpODP peptide family without and with cell-penetrant amino-acid TAT sequences. The p53-oligomerization-doman peptides (mtp53ODP) may be any one of:

SEQ ID NO: 1
GEYFTLQIRGRERFEMFRELNEALELK
SEQ ID NO: 2
GEYFTLQIRGRERFEMFRELNEALELKDAQAG
SEQ ID NO: 3
RKKRRQRRGEYFTLQIRGRERFEMFRELNEALELK
SEQ ID NO: 4
RKKRRQRRGEYFTLQIRGRERFEMFRELNEALELKDAQAG
SEQ ID NO: 5
KRALPNNTSSSPQPKKKPLDGEYFTLQIRGRERFEMFRELNEALELK
SEQ ID NO: 6
KRALPNNTSSSPQPKKKPLDGEYFTLQIRGRERFEMFRELNEALELKDAQ
AG
SEQ ID NO: 7
GRKKRRQRRGEYFTLQIRGRERFEMFR
SEQ ID NO: 8
GRKKRRQRRRGEYFTLQIRGRERFEMFRELNEALELK

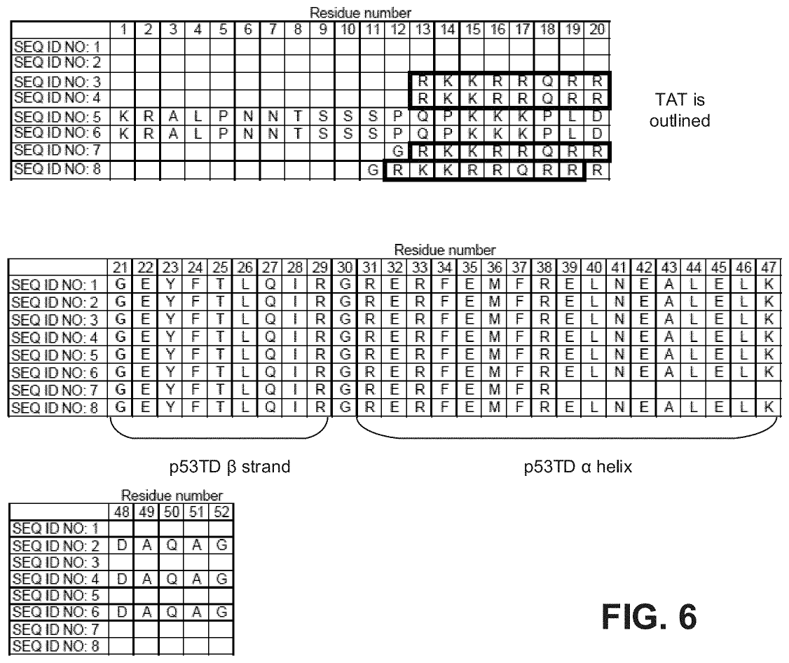

or a fragment thereof, or a variant of SEQ ID NOS: 1-8. The variant may comprise any minimal p53 oligomerization-domain motif sequence. FIG. 6 depicts a sequence alignment wherein SEQ ID NOs: 1-8 are aligned by the first residue in the p53TD β strand (e.g. residue 21 in SEQ ID NO: 3). The peptide may be conjugated to a fatty acid, i.e. myristoylated or linked to a carrier peptide. The carrier penetrating peptide can be any TAT variant or p53 nuclear localization domain variant, or transportan or polyarginine amino acid sequence. The mtpODP generally has fewer than forty residues. The peptide may be part of a pharmaceutical formulation, which may include a pharmaceutically acceptable excipient.

These mtp53ODP peptides can be used for other forms of PET imaging and therapeutic targeting including adding radioligands following pre-targeting with the D-mtp53ODP peptides and creating D-mtp53ODP that are “stapled” to increase the peptide stability. These methods would have the advantage of being able to use stabled peptides and radioligands with short half-lives and inhibit the exposure time of patients and the hospital workforce.

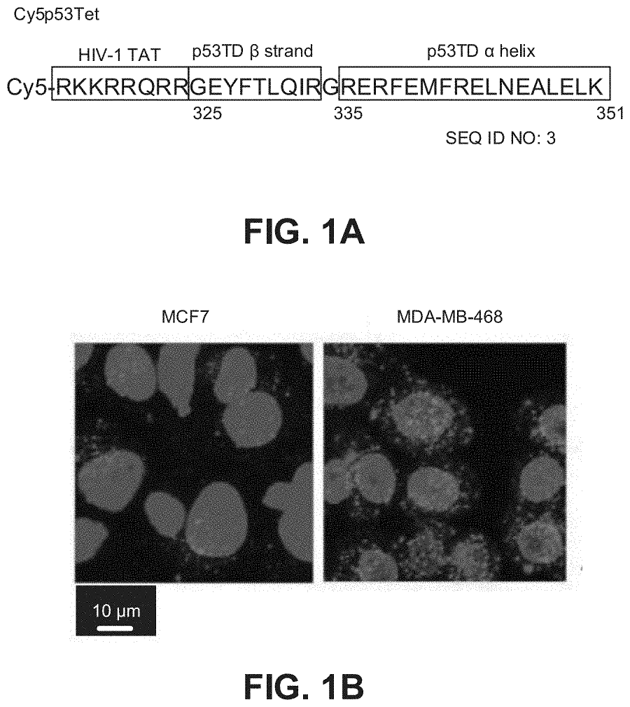

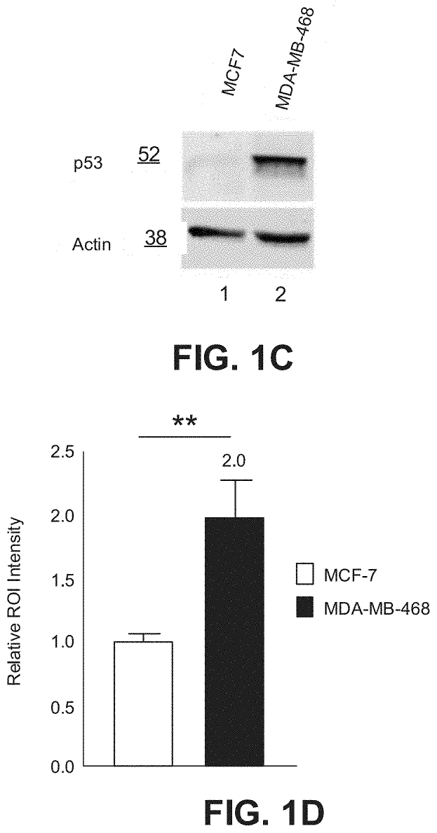

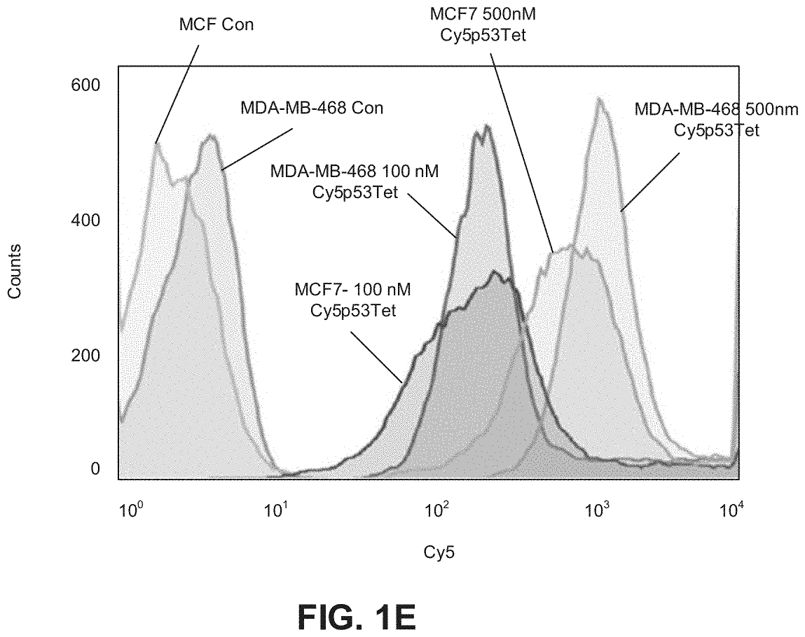

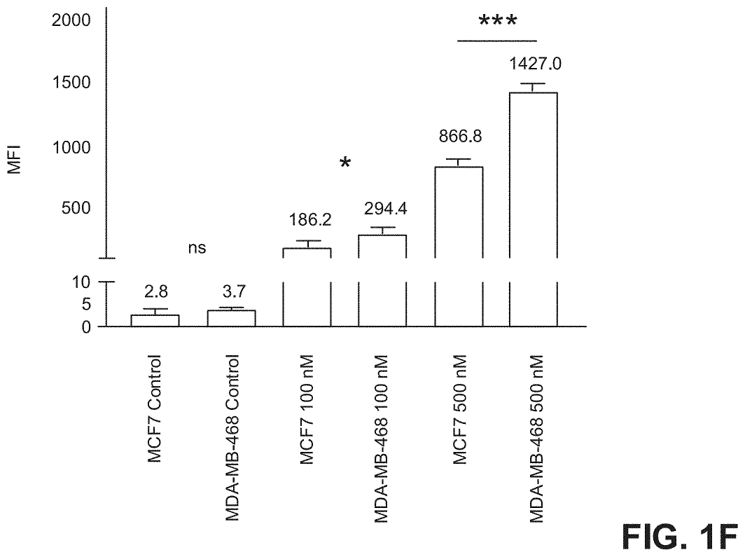

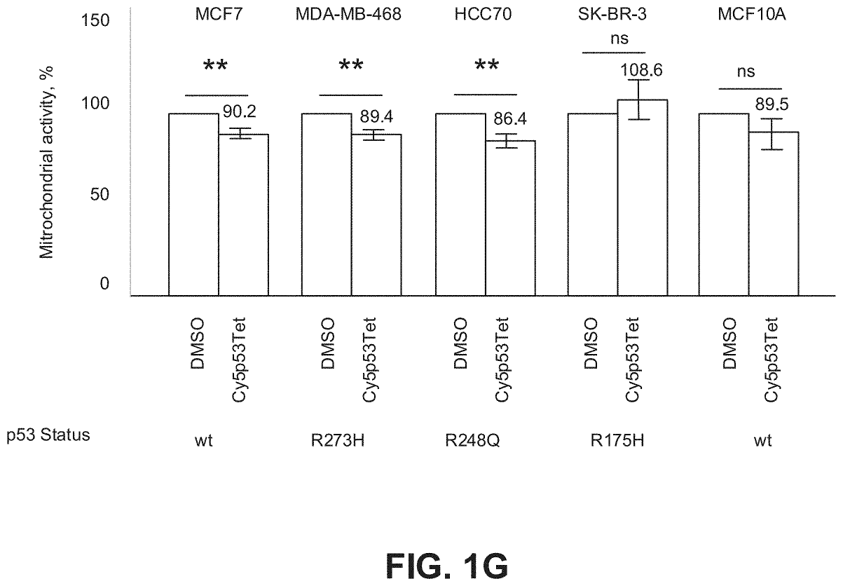

Referring to FIG. 1A, a mtp53ODP (SEQ ID NO: 3 with Cy5, referred to herein as Cy5p53Tet) was designed to monitor the expression of mtp53 in the nuclei of cancer cells contained three components: (1) a p53TD (residues 325-351) sequence to facilitate the recognition of the TD of mtp53, (2) a Cy5 fluorophore to enable NIRF imaging, and (3) an N-terminal HIV-1 TAT nuclear localization sequence to ensure the correct subcellular targeting (FIG. 1A).

In vitro uptake, specificity, and toxicity assays were performed to gauge the potential of mtp53ODPs as imaging agents. Live cell imaging was performed to compare the ability of Cy5p53Tet to stain ER+MCF7 breast cancer cells that express wtp53 and MDA-MB-468 TNBC cells which express stable missense mtp53 R273H. Cy5p53Tet was anticipated to detect both wtp53 and mtp53, but because of the higher stability of mtp53, MDA-MB-468 cells were predicted to have a higher signal. The intensity of the Cy5p53Tet signal was clearly higher in the MDA-MB-468 cells compared to the MCF7 cells (FIG. 1B), a difference that correlated to the level of p53 expression as determined by Western blot (FIG. 1C). Merged fluorescent images of the cell staining with Cy5p53Tet and Hoechst revealed the significant nuclear localization of Cy5p53Tet in the MDAMB-468 cells (see Supplemental Illustration S1 in U.S. Provisional Patent application 63/186,409 for vehicle control and 3D images). Quantification of the uptake of Cy5p53Tet via Nikon Element analysis demonstrated a twofold higher uptake of the mtp53ODP in MDA-MB-468 cells compared to MCF7 cells (FIG. 1D). The Cy5p53Tet did not colocalize with the stable transfected cell GFP marker in the cytoplasm, as determined by confocal microscopy. However, after a 2 h incubation with Cy5p53Tet, the colocalization was clearly evident with the Hoechst-stained DNA (Supplemental Illustration S3 in U.S. Provisional application 63/186,409).

The cellular uptake of Cy5p53Tet in these two cell lines was also measured using flow cytometry. The data from flow cytometry of MCF7 and MDA-MB-468 treated with vehicle or Cy5p53Tet at 100 or 500 nM for 2 h showed increased uptake in the mtp53-expressing cells (FIG. 1E). The geometric mean fluorescence intensity (MFI) values showed that the uptake of Cy5p53Tet was significantly elevated in MDA-MB-468 cells with mtp53 compared to MCF7 cells with wtp53, with 1.58-fold and 1.65-fold more in the mtp53-expressing cells at 100 and 500 nM Cy5p53Tet, respectively (FIG. 1F).

Toxicity to normal cells and tissues represents an important barrier for drug delivery, and an advantage of cell penetrating peptide-based therapies is their low toxicity compared to most drug carriers. To evaluate the toxicity of Cy5p53Tet to breast cancer cells and normal breast mammary epithelial cells, the viability of cells was evaluated following peptide treatment (FIG. 1G). No significant reduction of mitochondrial activity was detected in nonmalignant human mammary epithelial cells MCF-10A with wtp53 after 24 h of treatment with 500 nM Cy5p53Tet. The breast cancer cells treated with 500 nM Cy5p53Tet demonstrated that mitochondrial activity reduced by 9.8% in MCF7 cells (wtp53), 10.6% in MDA-MB-468 cells (mtp53 R273H), and 13.6% in HCC70 (mtp53 R248Q). Overall, Cy5p53Tet exhibited no cytotoxicity to normal cells and low cytotoxicity to cancer cells in vitro which paved the way for subsequent in vivo investigations with the imaging agent.

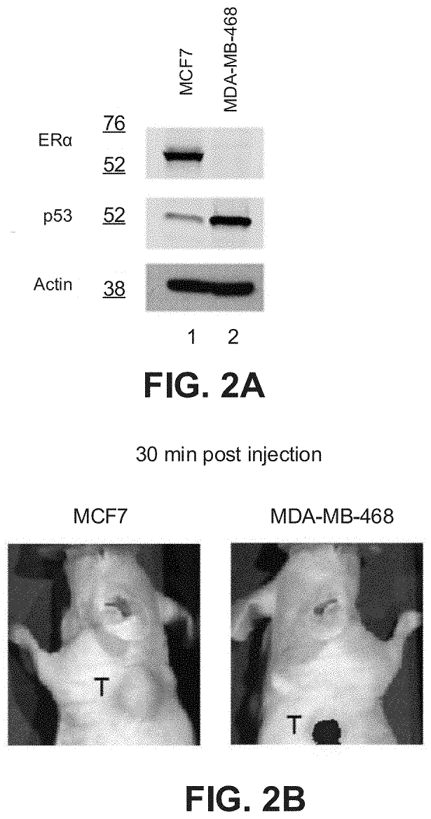

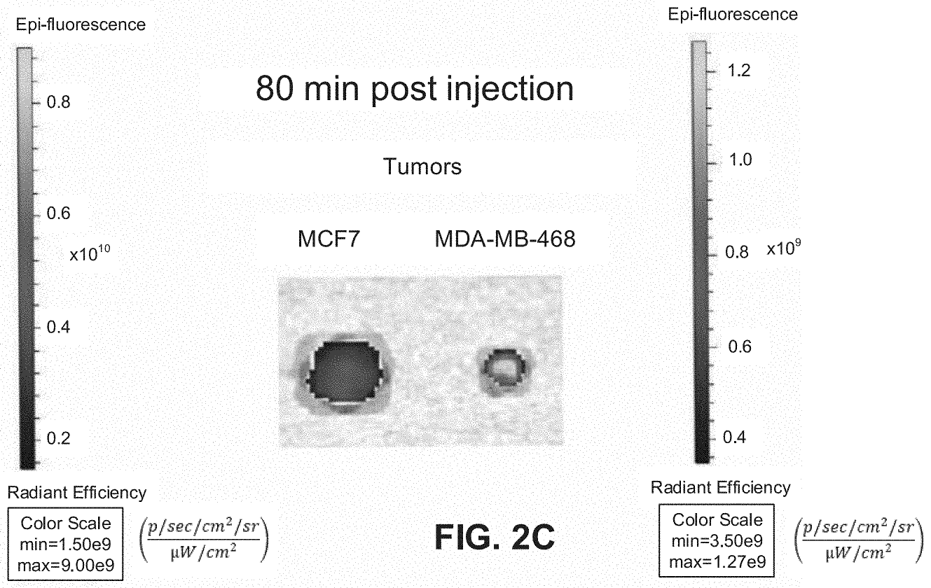

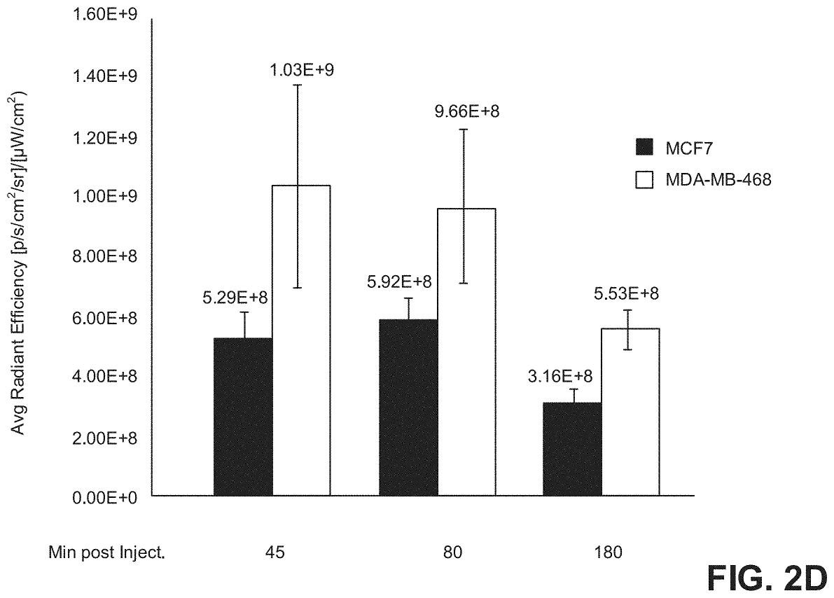

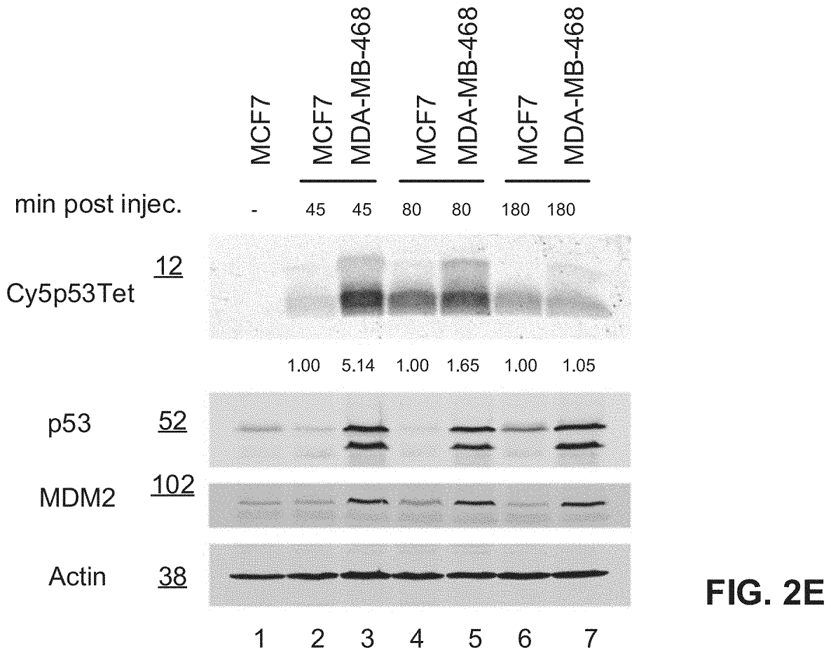

Cy5p53Tet is also useful for imaging of tumors with mtp53. In vivo NIRF imaging experiments were performed in mice bearing bilateral MCF7 and MDA-MB-468 xenografts that express wtp53 and mtp53 R273H (FIG. 2A to 2E). A Western blot confirmed the expression profiles of ER and p53 in the two cell lines prior to implantation, with the MCF7 cells exhibiting high levels of ER and low wtp53 and the MDA-MB-468 cells producing high levels of mtp53 (FIG. 2A). The NIRF signal was clearly observed in the MDA-MB-468 tumors, after the intravenous administration of Cy5p53Tet, whereas no signal was present in the MCF7 (FIG. 2B). At 40, 80, and 180 min after injection, mice were sacrificed, and the xenografts were resected and imaged ex vivo (FIG. 2C). At each time point, the MDAMB-468 mtp53-expressing tumors exhibited higher radiant efficiency than the MCF7 xenografts (FIG. 2D). More specifically, the ratios of the Cy5 signal in the MDA-MB-468 xenografts to that in the MCF7 tumors at 40, 80, and 180 min were 1.95:1, 1.63:1, and 1.75:1 respectively (FIG. 2D).

The accretion of Cy5p53Tet in tumor tissue was investigated by extracting proteins from the xenografts excised at 40, 80, and 180 min after injection and examined them on a 12% SDS polyacrylamide gel (FIG. 2E). The amount of the Cy5 channel by a Typhoon FLA 7000 laser scanner and quantifying the signal intensity. In this analysis, the ratios of the Cy5 signal derived from the MDA-MB-468 cells to that in the MCF7 cells at 40, 80, and 180 min were 5.14:1, 1.65:1, and 1.05:1 respectively. The expression levels of p53 and MDM2 following Cy5p53Tet treatment were probed by Western blot. The levels of both oncogenic proteins mtp53 and MDM2 were higher in the TNBC MDA-MB-468 cells (FIG. 2E). Interestingly, in MCF7 cells during the Cy5p53Tet uptake, wtp53 and MDM2 levels demonstrated the classic oscillation pattern (FIG. 2E, lanes 2, 4, and 6). This is logical, as cellular wtp53 is an unstable protein, with a half-life ranging from 5 to 30 min in the absence of an activation signal. The short half life is due to MDM2, a transcriptional target of wtp53 that acts as an E3 ubiquitin ligase that binds to p53 for proteosomal degradation. Oscillations in p53 and Mdm2 protein levels are required to keep wtp53 levels low. Taken together, the elevated uptake of Cy5p53Tet in the mtp53-expressing tumors of the mice indicates that Cy5p53Tet preferentially targets tumors expressing mtp53. The permeation properties of Cy5p53Tet hold great potential as in vivo TNBC detection agents and could be a delivery vehicle for cancer treatments.

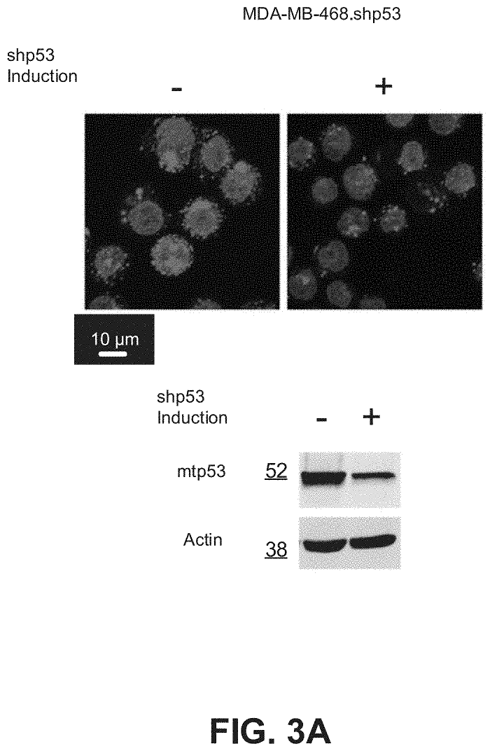

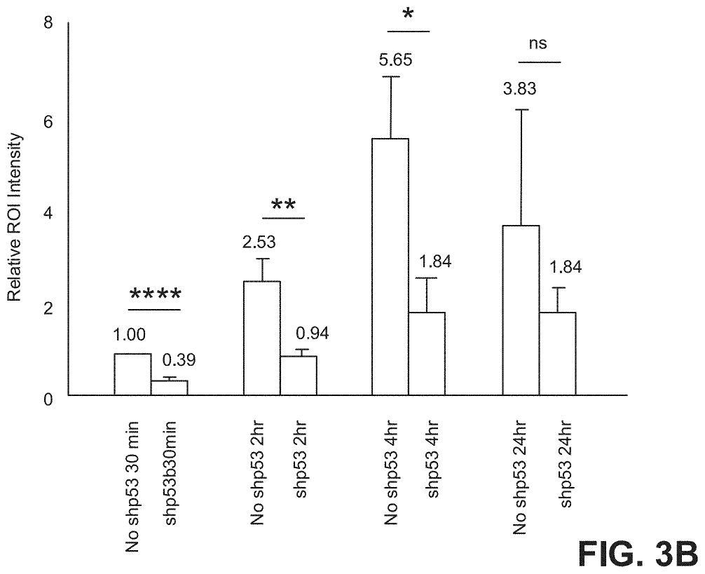

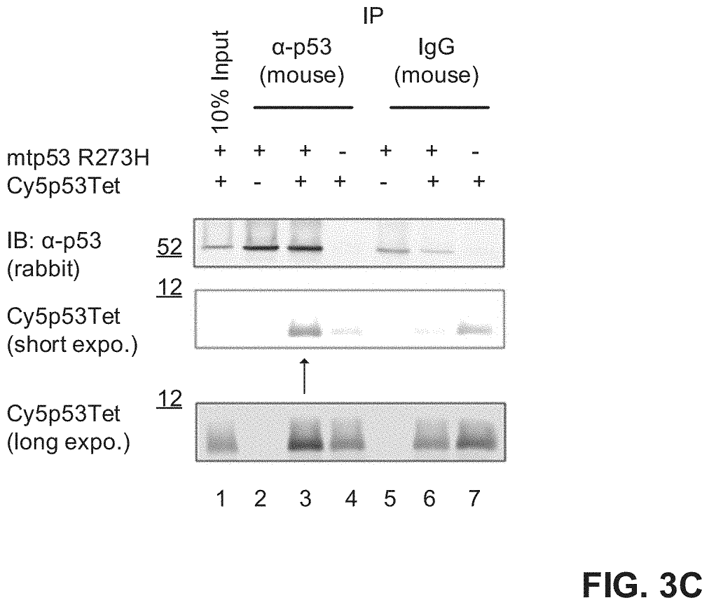

The specificity of the interaction between Cy5p53Tet and mtp53 R273H was further investigated by studying the uptake of the peptide in MDA-MB-468 cells with and without the depletion of mtp53 (FIG. 3A upper panel and Supplemental Illustrations S2 and S3 in U.S. Provisional application 63/186,409). To this end, a miR30-based doxycycline-inducible shRNA mtp53 knockdown cell line MDA-MB-468.shp53 was employed. The cells were incubated with 500 nM Cy5p53Tet for 30 min, 2 h, 4 h, and 24 h under both knockdown and control conditions (FIG. 3B). The imaging of the cells with the shRNA-mediated knockdown of the mtp53R273H message and protein also showed a corresponding reduction in the Cy5p53Tet uptake (Supplemental Illustration S3 in U.S. Provisional application 63/186,409). Cy5p53Tet could be detected as early as 30 min and was localized predominantly to the nucleus. The signal from the peptide increased up to 4 h, after which it was sustained until its decrease after 24 h. Most importantly, the depletion of mtp53 clearly correlates with reduced uptake of Cy5p53Tet (FIG. 3A, FIG. 3B, and Supplemental Illustrations S2 and S3 in U.S. Provisional application 63/186,409). More specifically, the relative region of interest intensity values showed a statistically significant reduction of 61, 63, and 67% of the Cy5p53Tet signal at 30 min, 2 h, and 4 h, respectively. By 24 h, however, the overall Cy5p53Tet signal was greatly reduced, and no statistically significant difference could be observed between the knockdown and control cells (FIG. 3B).

To determine if the Cy5p53Tet interacts with the p53TD, we first evaluated the p53TD and Cy5p53Tet complex using the protein-peptide globe docking method CABS-dock and obtained a high-quality prediction (see Supplemental Illustration S4 in U.S. Provisional application 63/186,409). To experimentally determine if Cy5p53Tet binds to mtp53 R273H, a co-immunoprecipitation assay was performed using purified mtp53 mixed with Cy5p53Tet in a molar ratio of 1:1 (FIG. 3C). The co-immunoprecipitation was carried out using anti-p53 antibody or nonspecific IgG. We found that mtp53 R273H was enriched by the anti-p53 antibody and not enriched by immunoprecipitation with IgG. Cy5p53Tet was significantly pulled down with mtp53 R273H in the anti-p53 antibody sample (see arrow), but not by the normal IgG sample (FIG. 3C).

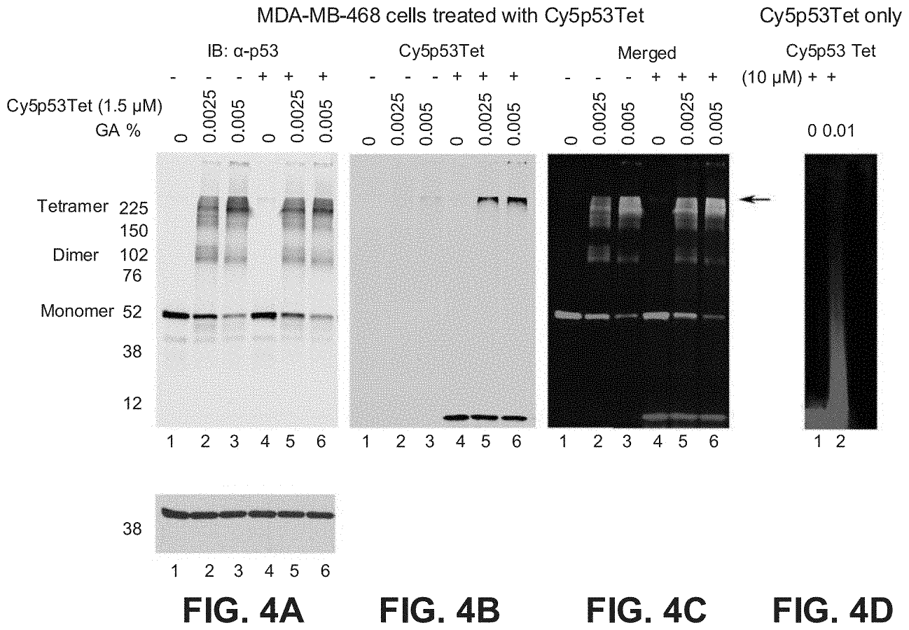

Wtp53 can form a tetramer, and its oligomerization regulates its transcriptional activity. Tetramerization is important for both wtp53 and mtp53, as both are preferentially degraded by MDM2 when present as dimers rather than tetramers. The oligomerization states of mtp53 were examined and the interactions were investigated by Cy5p53Tet and mtp53 using glutaraldehyde (GA) cross-linking assays (FIG. 4A to FIG. 4D). MDA-MB-468 cells were treated with vehicle or 1.5 μM of Cy5p53Tet for 4 h. Whole-cell lysates were cross-linked with glutaraldehyde concentrations of 0, 0.0025, or 0.005%, and the samples were analyzed by Western blot to detect p53 oligomerization forms (FIG. 4A). In cells without glutaraldehyde, monomers of mtp53 were detected; at 0.0025% glutaraldehyde, monomers, dimers, and tetramers were visible; and at 0.005% glutaraldehyde, the predominant form was tetramers. The presence of Cy5p53Tet-containing mtp53 polypeptides was evaluated using the Cy5 channel (FIG. 4B). Cy5p53Tet was detected at a low molecular weight: <12 kDa in the absence of glutaraldehyde (FIG. 4B, lane 4). Remarkably, in the presence of glutaraldehyde, Cy5p53Tet was observed at the molecular weight larger than the mtp53 tetramer: >225 kDa (FIG. 4B, lanes 5 and 6). The merged mtp53 and Cy5p53Tet images demonstrated that this high-molecular-weight species (yellow, indicated with arrow) was a p53/Cy5p53Tet complex. As a control, the glutaraldehyde cross-linking of Cy5p53Tet was examined without mtp53 and found no such signal (FIG. 4D). Similar results were observed in MDA-MB-468 cells treated with Cy5p53Tet for 2 or 4 h with higher glutaraldehyde levels (Supplemental Illustration S5 in U.S. Provisional application 63/186,409). Ultimately, the high molecular-weight complex that was >225 kDa is larger than the p53 tetramer detected in the MDA-MB-468 cells treated with Cy5p53Tet and could represent the hetero-tetramerization of mtp53 and Cy5p53Tet.

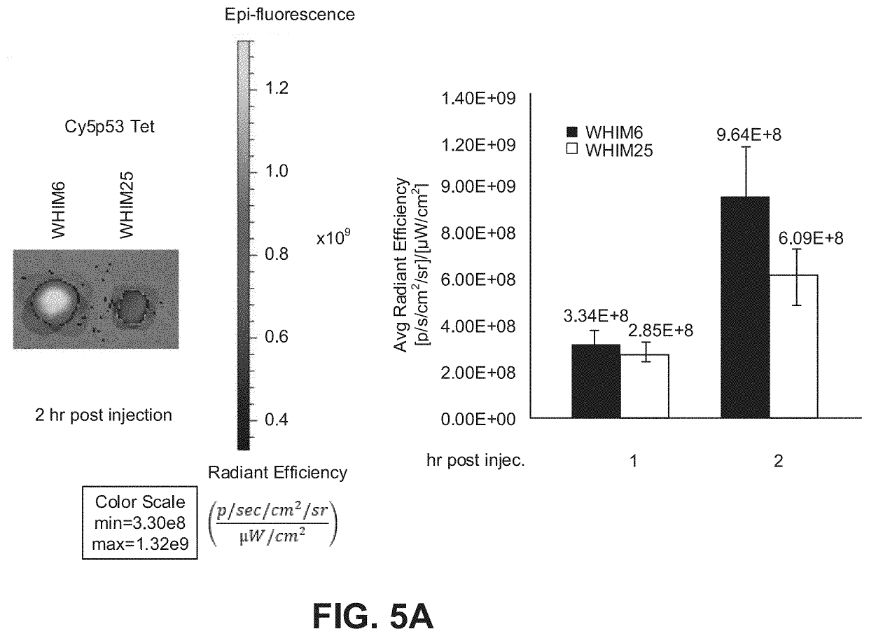

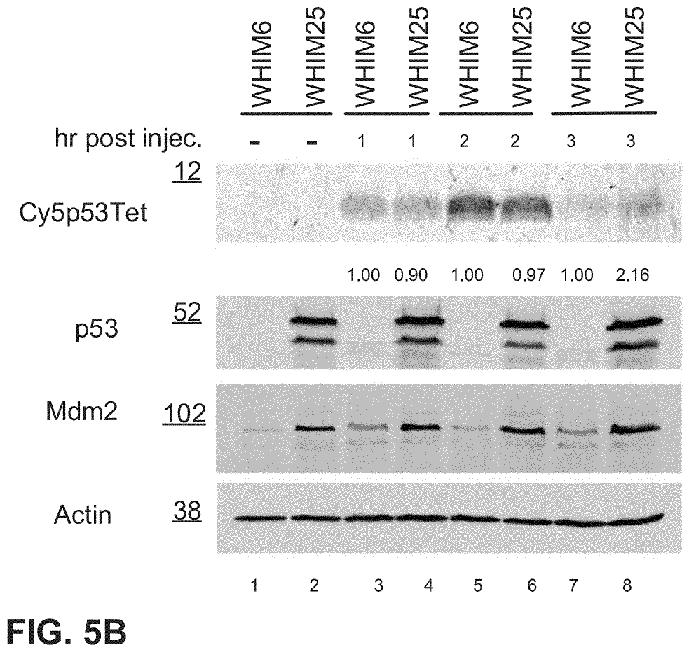

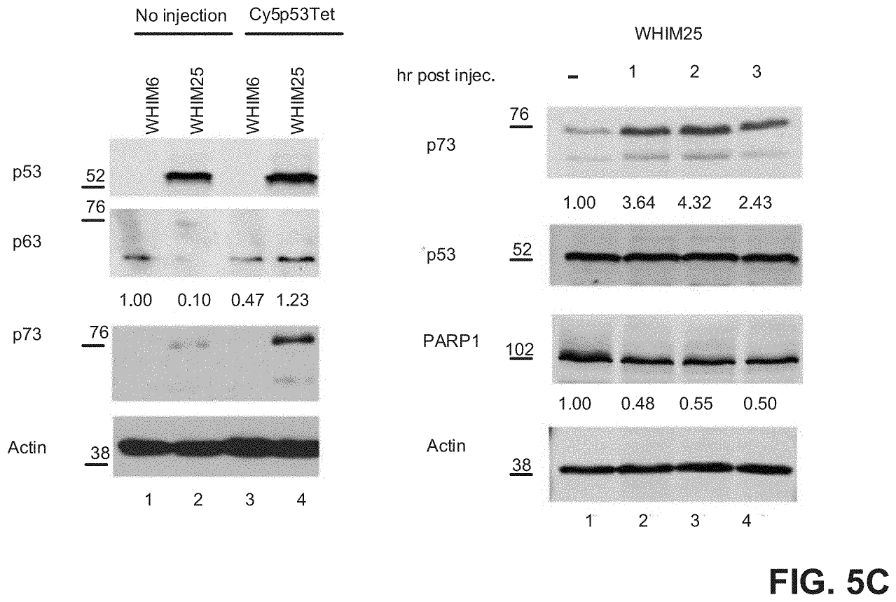

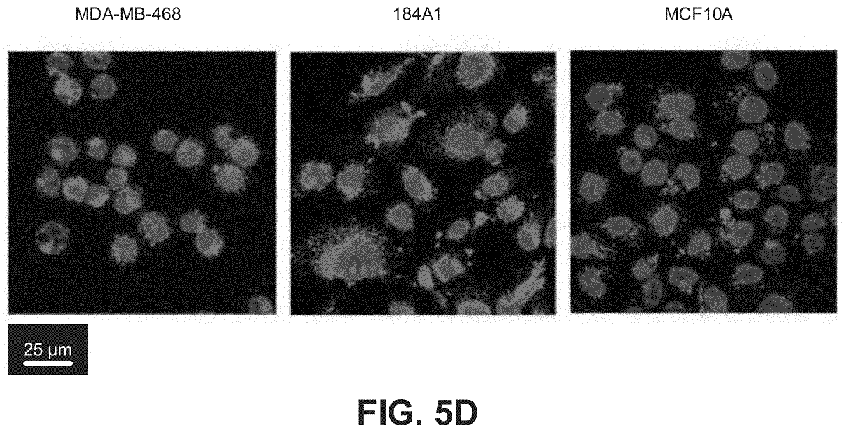

PDX models have been used in translational cancer research to validate the mechanisms that link specific biomarkers to treatment efficacy that could make clinical decisions. The coexpression of mtp53 and PARP1 proteins can be biomarkers for companion diagnostics using PDX models with mtp53. Cy5p53Tet peptide's uptake was tested and its effect on the modulation of wtp53 and mtp53 signaling in TNBC PDX WHIM6 expressing wtp53 and WHIM25, which expresses mtp53 R273H (FIG. 5A, FIG. 5B and FIG. 5C). First, WHIM6/WHIM25 bilateral PDX tumor-bearing NSG mice were tail vein injected with 10 nmol of Cy5p53Tet peptide to study its delivery efficacy (FIG. 5A). Then, the WHIM6 and WHIM25 tumors were removed at 1, 2, and 3 hr post-injection. Uptake of the Cy5p53Tet peptide in WHIM6 and WHIM25 tumors was assessed on an in vivo epifluorecent imaging system (IVIS). The Cy5p53Tet peptide was detected in both TNBC PDX tumors; however, there was a higher Cy5 fluorescence signal in the tumor site of WHIM6 compared to tumor site of WHIM25 (FIG. 5A, represent picture of 2 hr post injection). The histogram graph demonstrated the average radiant efficiency of the Cy5p53Tet peptide uptake by WHIM6 and WHIM25 (FIG. 5A right panel). The Cy5p53Tet peptide signal was investigated in PDX tumor tissue by extracting proteins from the WHIM6 and WHIM25 tumors post-injection with the Cy5p53Tet peptide at 1 hr, 2 hr, and 3 hr, and the Cy5p53Tet peptide effects were characterized in PDX models (FIG. 5B). Samples were run on 12% polyacrylamide gel and the Cy5p53Tet peptide accumulated in tumors was scanned at Cy5 channel and quantified. A miniscule difference of uptake of the Cy5p53Tet peptide was observed between WHIM6 and WHIM25 tumor tissue. However, the retention of Cy5p53Tet was longer in mtp53 R273H WHIM25 than in wtp53 WHIM6 models (FIG. 5B compare lane 8 to 7). The Cy5 fluorescence intensity of the tumor's tissue in WHIM25 versus WHIM6 at 1 hr, 2 hr, and 3 hr had a ratio of 0.90:1, 0.97:1, and 2.16:1 respectively. In addition, p53 and MDM2 protein levels were checked upon Cy5p53Tet peptide treatment in PDX tumors. Significantly high levels of mtp53 proteins were recorded in WHIM25 but undetectable levels of wtp53 in WHIM6. Both mtp53 and wtp53 protein levels were not affected by the Cy5p53Tet peptide treatment. Interestingly, MDM2 protein levels were higher in WHIM25 than in WHIM6 tumors.

Given the high degree of structural similarity of the tetramerization domain shared by the p53 family members, p63, and p73, the Cy5p53Tet peptide is believed to bind to TD of p63 or p73 and cause the Cy5p53Tet peptide accumulation in WHIM6 tumors. The p63 and p73 protein levels were examined in WHIM6 and WHIM25 tumors by western blot (FIG. 5C left panel). A higher level of p63 was detected in WHIM6 than in WHIM25 tumors (FIG. 5C left panel, compare lane 1 to 2). p73 protein levels were very low in both WHIM6 and WHIM25 tumors. Increased p73 protein levels were notably found after the Cy5p53Tet peptide treatment in mtp53 WHIM25 (FIG. 5C left panel, compare lane 4 to 2), but not in wtp53 WHIM6 (FIG. 5C left panel, compare lane 3 to 1). Mutant p53 interacts with p73 resulting in the inhibition of p73 dependent apoptosis and chemosensitivity. Antitumor effects via the upregulating of p73 and disrupting its interaction with mutant p53 has been reported. To further investigate whether the Cy5p53Tet peptide might inhibit gain of function of mutant p53 activity, p73 and PARP1 protein levels were tested in mtp53 WHIM25 tumor samples (FIG. 5C right panel). A high expression level of PARP1 was found in triple-negative breast cancer in the TCGA database. Remarkably, increased p73 protein and deceased PARP1 protein levels were detected in response to the Cy5p53Tet peptide in WHIM25 tumor samples (FIG. 5C right panel, compare lane 2, 3& 4 to lane 1).

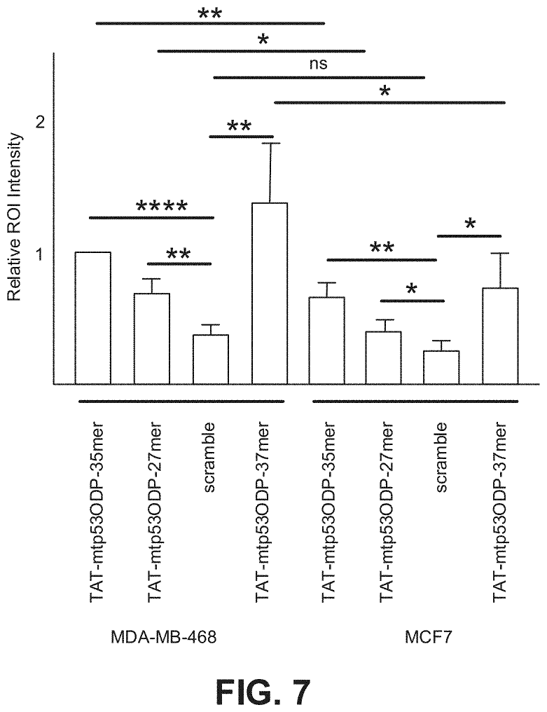

Live cell imaging staining was also performed on second-generation peptides based on Cy5-conjugated SEQ ID NO: 7 and SEQ ID NO: 8. FIG. 7 depicts the results of live cell imaging staining of MDA-MB-468 and MCF7 cells with 4 h incubation of 500 nM Cy5 conjugated first (TAT-mtp53ODP-35mer, SEQ ID NO: 3) and second-generation (TAT-mtp53ODP-27mer (SEQ ID NO: 7) and Cy5-conjugated TAT-mtp53ODP-37mer (SEQ ID NO: 8)) after 4 h incubation. Cells were imaged by confocal microscopy. A scrambled peptide was included as a control. The TAT-mtp53ODP-27mer (SEQ ID NO: 7) showed reduced activity relative to TAT-mtp53ODP-35mer (SEQ ID NO: 3). The TAT-mtp53ODP-37mer (SEQ ID NO: 8) showed enhanced activity relative to TAT-mtp53ODP-35mer (SEQ ID NO: 3).

Materials and Methods

Materials. The mutant p53 oligomerization domain peptide (mtp53ODP) called Cy5p53Tet was purchased from JPT peptide (Germany) at a purity >95%. The mtp53ODP is 35 amino acids long with an N-Terminal Cy5 fluorophore conjugation: H-CysCy5-RKKRRQRRGEYFTLQIRGRERFEMFRELNEALELK-OH (SEQ ID NO: 3).

Solvents and reagents including dimethyl sulfoxide (DMSO), glutaraldehyde (GA), and anti-β-actin antibody (Cat #A2066) were obtained from Sigma-Aldrich. Anti-p53 antibody (DO-1, Cat #sc-126), anti-PARP1 antibody (Cat #sc-7150), and normal mouse IgG (Cat #sc-2025) were purchased from Santa Cruz. Magnetic beads were purchased from Cell Signaling. Anti-MDM2 antibody (Cat #AF1244) was obtained from the R&D System.

Ethics. All animal experiments were done in accordance with protocols approved by the Institutional Animal Care and Use Committees (IACUC) of Hunter College, Weill Cornell Medical College, and Memorial Sloan Kettering Cancer Center and followed the National Institutes of Health guidelines for animal welfare.

Cell Culture. Human breast cancer cell lines MCF7, MDAMB-468, MDA-MB-231, HCC70, and SK-BR-3 and normal human mammary epithelial cell MCF10A were purchased from American Type Culture Collection (ATCC). We have authenticated all the cell lines by short tandem repeat technology (Genetica DNA Laboratories). Cells were tested for Mycoplasma using the Universal Mycoplasma Detection Kit from ATCC. Cells were maintained at 5% CO₂ in a 37° C. humidified incubator. MCF7, MDA-MB-468, MDA-MB-231, and HCC70 cells were grown in DMEM (Invitrogen) and supplemented with 10% FBS (Gemini). SK-BR-3 cells were cultured in McCoy's 5a Medium and supplemented with 10% FBS (Gemini). MCF10A cells were grown in MEGM Mammary Epithelial Cell Growth Medium SingleQuots Kit without gentamycin-amphotericin B mix (Lonza) with 100 ng/mL cholera toxin. All cells were supplemented with 50 U/mL penicillin, 50 μg/mL streptomycin (Mediatech), and 5 μg/mL plasmocin (InvivoGen). MDA-MB-468 shp53 cells generated with mir30 short hairpin RNA can induce knockdown of mtp53 with 8 μg/mL doxycycline for 7 days.

Cy5p53Tet Cellular Uptake by Live Cell Imaging. Cells were seeded at 2×105 per well in a 12-well glass bottom plate 1 day before imaging (MatTek). Cells were incubated with 100 or 500 nM Cy5p53Tet at 37° C. for the indicated time. Cy5p53Tet was then removed, and the cells were washed three times with phosphate-buffered saline (PBS) at room temperature and costained with 1 μg/mL Hoechst 33342 (Thermo-Fisher) in PBS for 5 min. Z-stack images of stained cells were taken by confocal microscopy using a Nikon A1 confocal microscope with a 60× objective.

In Vitro Cy5p53Tet Cellular Uptake by Flow Cytometry. Fluorescence-activated cell-sorting (FACS) was used to determine the cellular uptake of Cy5p53Tet. MCF7 and MDA-MB-468 cells were seeded in six-well plates at a density of 5×10⁵ cells/well and incubated at 37° C. overnight. On the following day, media were replaced with fresh media containing vehicle control or 100 or 500 nM Cy5p53Tet and further incubated at 37° C. for 2 h. Cells were washed two times with PBS and trypsinized at 37° C. for 5 min. Trypsin was neutralized by adding media, and the cell suspension was spun down. Cell pellets were washed with PBS and resuspended in PBS. FACS was performed on a FACScan (BD Biosciences), processing 2×10⁴ events for each sample.

Peptidecytotoxicity Assay. A total of 1.25×10⁵ cells were seeded in a 12-well plate the day before and grown at 37° C. Cells were treated with 500 nM Cy5p53Tet for 24 h, and 0.1 mL MTT solution [5 mg mL⁻¹ (3-(4, 5-dimethylthiazolyl-2)-2, 5-diphenyltetrazolium bromide] was added to the cells and incubated at 37° C. for 1 h. The cells were then resuspended in 0.04 N hydrochloric acid diluted in isopropanol and incubated in the dark on a shaker for 5 min at room temperature. The absorbance was quantified at 550 nm, and the background absorbance was subtracted at 620 nm.

Co-immunoprecipitation Assay. Magnetic beads were used for co-immunoprecipitation assays, and 50 μL of the bead suspension was placed in each sample. The beads were washed twice with 1×PBS-0.1% Tween-20 by vortexing for 10 s. The beads were resuspended in 1×PBS-0.1% Tween-20 with either 1 μg of anti-p53 DO1 antibody or 1 μg of normal mouse IgG at a final volume of 100 μL. The tubes were incubated at room temperature for 10 min with continuous mixing. The beads were washed three times with 1×PBS-0.1% Tween-20, and 1 μg of purified mtp53 R273H and 100 ng of Cy5p53Tet peptide were incubated with the immobilized antibody at 4° C. for 150 min with constant rotation. Beads were pelleted and then washed with 1 mL 1×PBS-0.1% Tween-20 four times at room temperature. Bound proteins were eluted by incubating in 2×SDS Laemmli sample buffer containing 0.2 M DTT, heated at 95° C. for 10 min, and loaded on 15 or 10% polyacrylamide gel.

Protein Extraction. Whole-cell extraction and protein extraction from xenograft models were conducted as previously described.

Glutaraldehyde Cross-Link Assay. Cells were treated with vehicle control or Cy5p53Tet for 2 or 4 h and lysed with phosphate lysis buffer (PBS, 10% glycerol, 10 mM EDTA, 0.5% NP-40, 0.1 M KCl, 1 mM PMSF, 8.5 μg/mL aprotinin, 2 μg/mL leupeptin, and phosphatase inhibitor cocktail). Glutaraldehyde was added to 100 μg of lysate to final concentrations of 0.0025, 0.005, or 0.01%. After incubating with rotation for 20 min at room temperature, the reactions were stopped by adding 2×SDS Laemmli sample buffer containing 0.2 M DTT, the samples were heated for 5 min at 100° C., and 25 μg of sample was resolved by 8% SDS-PAGE.

NIRF Imaging of Cy5p53Tet in Mice Bearing Bilateral MCF7/MDA-MB-468 Xenografts. Female athymic nude mice (6-10 weeks old, 01B74-Athymic NCr-nu/nu;) were obtained from Charles River Laboratories. Animals were supplemented with 17β-estradiol with a dose of 0.72 mg/pellet (60-day release) into the neck 7 days before MCF7 cells were subcutaneous implanted. 5×10⁶ cells/mouse MCF7 cells were suspended in 100 μL of 1:1 media/matrigel basement membrane matrix (Corning) and injected subcutaneously on the left flank of each mouse (n≥3/group). After 4 weeks, 5×10⁶ cells/mouse MDA-MB-468 cells were subcutaneously implanted in the right flank of the mouse in 100 μL of 1:1 media/matrigel basement membrane matrix. Imaging experiments were performed when the tumors reached a volume of ˜50-250 mm³ (after approximately 3 weeks). Cy5p53Tet (10 nmol) was injected into the tail vein of each mouse. Prior to in vivo imaging, the mice were anesthetized with 1.5-2.0% isoflurane (Baxter Healthcare). Images were collected using an IVIS Spectrum (Perkin Elmer) 12 min, 30 min, and 3 h following the administration of Cy5p53Tet. Epifluorescence exposure time on each side was identical, with multiple exposures ranging from 0.2 to 2 s. Fluorescence imaging was carried out with excitation and emission wavelengths of 640 and 680 nm, respectively. Animals were sacrificed 40 min, 80 min, or 3 h after the injection of Cy5p53Tet, and epifluorescence images of the excised MCF7 and MDA-MB-468 xenografts were obtained using the same condition as mentioned above. Semiquantitative analysis of the Cy5p53Tet signal was conducted by measuring the average radiant efficiency [p/s/cm²/sr]/[μW/cm²] in regions of interest.

Statistical Analysis. Statistical analyses were conducted in Graphpad Prism 7. Results are expressed as mean+SEM. Statistical significance for hypothesis testing was performed by two-tailed Student's t-test of unknown variance.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A composition of matter, the composition of matter comprising:
a peptide with a formula of X-mtp53ODP wherein X is selected from a group consisting of (1) a detection agent, (2) a DNA-damaging agent and (3) combinations thereof, wherein X is covalently bonded to mtp53ODP which is a peptide selected from a group consisting of:
(SEQ ID NO: 1)
GEYFTLQIRGRERFEMFRELNEALELK;
(SEQ ID NO: 2)
GEYFTLQIRGRERFEMFRELNEALELKDAQAG;
(SEQ ID NO: 3)
RKKRRQRRGEYFTLQIRGRFRFEMFRELNEALELK;
(SEQ ID NO: 4)
RKKRRQRRGEYFTLQIRGRERFEMFRELNEALELKDAQAG;
(SEQ ID NO: 5)
KRALPNNTSSSPQPKKKPLDGEYFTLQIRGRERFEMFRELINEALELK;
(SEQ ID NO: 6)
KRALPNNTSSSPQPKKKPLDGEYFTLQIRGRERFEMFRELNEALELKDAQ
AG;
(SEQ ID NO: 7)
GRKKRRQRRGEYFTLQIRGRERFEMFR;
and
(SEQ ID NO: 8)
GRKKRRQRRRGEYFTLQIRGRERFEMFRELNEALELK.

2. The composition of matter as recited in claim 1, wherein X is a detection agent.

3. The composition of matter as recited in claim 1, wherein X is a radioligand.

4. The composition of matter as recited in claim 1, wherein X is DNA-damaging agent.

5. The composition of matter as recited in claim 1, wherein X is a fluorophore.

6. The composition of matter as recited in claim 1, wherein X is a cyanine fluorophore.

7. The composition of matter as recited in claim 1, wherein X is a Cy5 fluorophore.

8. A method of administering an agent to a patient, the method comprising: administering to a patient the agent recited in claim 1.

9. The composition of matter as recited in claim 1, wherein the peptide is SEQ ID NO: 3.

10. The composition of matter as recited in claim 9, wherein X is a fluorophore.

11. The composition of matter as recited in claim 9, wherein X is a cyanine fluorophore.

12. The composition of matter as recited in claim 9, wherein X is a Cy5 fluorophore.

13. The composition of matter as recited in claim 1, wherein the peptide is SEQ ID NO: 7.

14. The composition of matter as recited in claim 13, wherein X is a fluorophore.

15. The composition of matter as recited in claim 13, wherein X is a cyanine fluorophore.

16. The composition of matter as recited in claim 13, wherein X is a Cy5 fluorophore.

17. The composition of matter as recited in claim 1, wherein the peptide is SEQ ID NO: 8.

18. The composition of matter as recited in claim 17, wherein X is a fluorophore.

19. The composition of matter as recited in claim 17, wherein X is a cyanine fluorophore.

20. The composition of matter as recited in claim 17, wherein X is a Cy5 fluorophore.
